(19) United States
(12) Patent Application Publication
(10) Pub. No.: US 2022/0189587 A1
(43) Pub. Date: Jun. 16, 2022

(54) GENERATION OF CODES FOR CHEMICAL STRUCTURES FROM NMR SPECTROSCOPY DATA

(71) Applicant: Bayer Aktiengesellschaft, Leverkusen (DE)

(72) Inventors: Djork-Arne CLEVERT, Berlin (DE); Jan Robin WINTER, Berlin (DE)

(73) Assignee: Bayer Aktiengesellschaft, Leverkusen (DE)

(21) Appl. No.: 17/644,527

(22) Filed: Dec. 15, 2021

(30) Foreign Application Priority Data
Dec. 16, 2020 (EP) 20214534.8

(51) Int. Cl. G16C 20/20; G06F 30/27; G16C 20/70

(52) U.S. Cl. CPC G16C 20/20; G16C 20/70; G06F 30/27

(57) ABSTRACT

A method of generating codes for chemical structures from NMR spectroscopy data comprises receiving spectroscopic data of a chemical compound, inputting the spectroscopic data into a first artificial neural network to generate molecular descriptors, receiving a molecular descriptor from the first artificial neural network, inputting the molecular descriptor a second artificial neural network to convert structure data of the chemical reference compounds to molecular descriptors and to convert the molecular descriptors back to the structure data, and receiving structure data of the chemical compound from the second artificial neural network.

### CROSS REFERENCE TO RELATED APPLICATIONS

This application claims priority benefit to European Application No. 20214534.8, filed Dec. 16, 2020, the disclosure of which is herein incorporated by reference in its entirety.

### FIELD OF THE DISCLOSURE

The present disclosure is concerned with the elucidation of the structure of chemical compounds. The present disclosure provides a method, a computer system, and a computer program product for generation of structure codes for chemical compounds from spectroscopic data of the chemical compounds.

### BACKGROUND OF THE DISCLOSURE

The chemical structure indicates the construction, at the molecular or ionic level, of a single (homogeneous) substance. It states how atoms, atomic groups, ions, bonds, and/or free electron pairs are arranged with respect to one another.

There are various methods of elucidating the structure of chemical compounds. For determination of the structure in liquids, gases, and amorphous solids, the following methods are particularly suitable: NMR spectroscopy, mass spectrometry, vibration spectroscopy, UV-vis spectroscopy, and x-ray absorption spectroscopy.

NMR spectroscopy (NMR: nuclear magnetic resonance) is a spectroscopic method of studying the chemical environment of individual atoms and the interactions with the neighboring atoms. The method is based on magnetic nuclear resonance, a resonant interaction between the magnetic moment of atomic nuclei of the sample present in a static magnetic field with a high-frequency alternating magnetic field. The only isotopes amenable to spectroscopy are those that have a non-zero nuclear spin and hence a magnetic moment in the ground state, for example 1H, ²D, ⁶Li, ¹⁰B, ¹¹B, ¹³C, ¹⁵N, ¹⁷O, ¹⁹F, ³¹P and ⁴³Ca.

¹³C NMR spectroscopy in combination with ¹H NMR spectroscopy is the most important method of elucidating the structure of organic molecules. Using a ¹³C spectrum and a ¹H NMR spectrum of a chemical compound, a chemist will typically be able to solve the structure of the chemical compound and hence determine/identify the chemical compound. A multitude of textbooks relating to the elucidation of the structure of organic chemical compounds have been published (see, for example, H. Duddeck, W. Dietrich: Strukturaufklärung mit moderner NMR-Spektroskopie [Structural Elucidation by Modern NMR Spectroscopy], Steinkopf-Verlag, 1988, ISBN: 978-3-642-97777-0; E. Pretsch et al.: Spektroskopische Daten zur Strukturaufklärung organischer Verbindungen [Spectroscopic Data for the Structure Determination of Organic Compounds], 4th edition, Springer-Verlag, 2001, ISBN: 978-3-540-41877-1).

Since structural elucidation using spectroscopic data is a sometimes time-consuming affair, it would be desirable to be able to accelerate this. It would be desirable to accelerate structural elucidation using spectroscopic data since this is sometimes a time-consuming affair.

This is achieved by the present invention.

The present disclosure provides, in some embodiments, a computer-implemented method comprising the steps of:
receiving spectroscopic data of a chemical compound,
inputting the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
receiving a molecular descriptor of the chemical compound from the first artificial neural network,
inputting the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
receiving structure data of the chemical compound from the second artificial neural network,
outputting and/or storing the structure data and/or information derived from the structure data.

In some embodiments, the present disclosure further provides a computer system comprising
an input unit,
a control and computation unit and
an output unit,
wherein the control and computation unit is configured to prompt the input unit to receive spectroscopic data of a chemical compound,
wherein the control and computation unit is configured to input the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
wherein the control and computation unit is configured to receive a molecular descriptor for the chemical compound from the first artificial neural network,
wherein the control and computation unit is configured to input the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
wherein the control and computation unit is configured to receive structure data of the chemical compound from the second artificial neural network,
wherein the control and computation unit is configured to prompt the output unit to output and/or to store the structure data received and/or information derived from the structure data.

In some embodiments, the present disclosure further provides a computer program product comprising a computer program which can be loaded into a memory of a computer system, where it prompts the computer system to:
receive spectroscopic data of a chemical compound,
input the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
receive a molecular descriptor of the chemical compound from the first artificial neural network,
input the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
receive structure data of the chemical compound from the second artificial neural network,
output and/or store the structure data and/or information derived from the structure data.

The invention will be more particularly elucidated below without distinguishing between the subjects of the invention (method, computer system, computer program product). On the contrary, the following embodiments are intended to apply analogously to all the subjects of the invention, irrespective of in which context (method, computer system, computer program product) they occur.

The present disclosure uses spectroscopic data of a chemical compound to produce structure data for the chemical compound.

The term “chemical compound” is understood to mean a pure substance consisting of atoms of two or more chemical elements, where the types of atom (by contrast to mixtures) are in a fixed ratio to one another. The numerical ratio of the atoms to one another is determined by chemical bonds between the atoms, and the ratio can be represented in an empirical formula.

The chemical compound is preferably an organic compound. An “organic compound” is a chemical compound comprising carbon-hydrogen bonds (C—H bonds). Preference is given to an organic compound, the molecules of which are formed solely from the following elements: carbon (C), hydrogen (H), oxygen (O), nitrogen (N), sulfur (S), fluorine (F), chlorine (CO, bromine (Br), iodine (I) and/or phosphorus (P).

The term “structure data” is understood to mean information from which the chemical structure of the chemical compound can be derived. The chemical structure defines the construction of the chemical compound at the molecular or ionic level. In other words: the chemical structure indicates how atoms, atomic groups, ions and bonds or free electron pairs are arranged with respect to one another.

The structure data are preferably information from which the complete chemical structure of a chemical compound can be inferred. Examples of such structure data are structure codes in which the chemical structure is encoded in the form of a machine-readable string of characters. Such a string of characters comprises characters of a defined set of characters, i.e. of a defined inventory of elements. Such elements (characters) may, inter alia, be the letters of an alphabet, numbers, but also other symbols, for example special characters, punctuation marks, the characters of the phonetic transcription of the IPA code (IPA: International Phonetic Alphabet) and/or of Braille script, pictograms of various kinds and/or control characters. Examples of known character sets are ASCII (American Standard Code for Information Interchange), EBCDIC (Extended Binary Coded Decimal Interchange Code) and Unicode. The mapping of a chemical structure of a chemical compound onto a structure code is usually unambiguous (for every chemical structure there is exactly one structure code) and reversible (the chemical structure can be reconstructed from the structure code, for example in the form of an image representation).

Examples of such structure codes are SMILES codes, InChI codes, CML codes and WLN codes. SMILES stands for Simplified Molecular Input Line Entry Specification. This is a chemical structure code in which the structure of any molecule is represented in highly simplified form as a string of ASCII characters. Multiple commercially available molecular editors can import SMILES strings and hence create two-dimensional and three-dimensional models of the molecules. The IUPAC International Chemical Identifier (InChI) is also a chemical structure code that enables unambiguous and reversible translation of a molecule to a standardized string of characters. A further example is the Chemical Markup Language (ChemML or CML), a data format based on XML (Extensible Markup Language) that can be used for representation of chemical formulae among other uses. A further example is Wiswesser Line Notation (WLN).

The IUPAC name of a chemical compound is also structure data for the purposes of the present invention.

“Spectroscopic data” are understood to mean information which is the result of a spectroscopic and/or spectrometric measurement method on the chemical compound. In spectroscopic measurement methods, there is an interaction between the chemical compound and electromagnetic radiation. The electromagnetic radiation is separated here according to a particular property such as wavelength and/or energy, and the interaction between the chemical compound and the electromagnetic radiation is analysed. The result of this analysis is typically a spectrum. The spectroscopic data are preferably one or more NMR spectra, infrared spectra and/or UV-vis spectra. Measurement methods not based on the interaction of the chemical compound with electromagnetic radiation are referred to in this description as spectrometric measurement methods; a known example is mass spectrometry. An overview of standard spectroscopic and spectrometric measurement methods is given, for example, by the following work: Encyclopedia of Spectroscopy and Spectrometry, J. C. Lindon, G. E. Tranter, D. W. Koppenaal, Elsevier 2017, volumes 1, 2, 3 and 4, ISBN 9780128105382, 9780128105368,9780128105375,9780128105511.

In a particularly preferred form, the spectroscopic data are one or more peak lists of a spectrum or multiple spectra of the chemical compound, more preferably at least one peak list from a ¹H NMR spectrum and/or at least one peak list from a ¹³C NMR spectrum.

An (NMR) spectrum of a chemical compound contains a number of local extremes (maxima and minima) that are generally referred to as peaks. These local extremes may be characterized by parameters such as position on the abscissa (in the case of NMR spectra, frequency or chemical shift), intensity, line width and/or volume. In order to analyze and hence to process local extremes in (NMR) spectra, the peaks in the spectrum are identified, their parameters are detected and they are stored as a descriptor in the form of the peak list. Commercial and freely available software tools are available for the automated analysis of spectra, the identification of peaks, the determination of the peak parameters and the production of a peak list (e.g. TopSpin, Mnova, INFOS, NMRPipe inter alia).

Most preferably, the spectroscopic data of the chemical compound comprise a peak list of a ¹³C NMR spectrum of the chemical compound, with all local extremes in the ¹³C NMR spectrum identified in the peak list using the following parameters: frequency or chemical shift, intensity, line width and/or peak integral.

The ¹³C NMR spectrum/the ¹³C NMR spectra of the chemical compound may be a ¹H broadband-decoupled spectrum, a DEPT spectrum (e.g. a DEPT-135 or a DEPT-90 spectrum), an APT spectrum, a ¹H off-resonance spectrum and/or a gated decoupling spectrum.

Details of the spectra mentioned and further spectra and of the recording of NMR spectra can be taken from the numerous textbooks on this topic (see, for example, C. Schorn, B. J. Taylor: NMR-Spectroscopy: Data Acquisition, 2nd edition, Wiley-Verlag 2005, ISBN: 978-3-527-60619-1; T.N. Mitchell, B. Costisella: NMR—From Spectra to Structures, Springer-Verlag 2013, ISBN: 9783662054345).

The generation of structure data of a chemical compound using spectroscopic data can be effected in multiple steps; in a first step, the spectroscopic data are used, with the aid of a first artificial neural network, to generate a molecular descriptor and, in a second step, the molecular descriptor, with the aid of a second artificial neural network, is used to generate structure data.

The molecular descriptor, like the structure data and especially the chemical structure code too, is an unambiguous representation of the chemical structure of the chemical compound: it is possible to generate exactly one molecular descriptor from a chemical structure (represented by the structure data), and to use the molecular descriptor to generate exactly one chemical structure (represented by the structure data). While the structure data, especially the chemical structure code, typically has a variable length (of the string of characters), the molecular descriptor has a fixed size, i.e. a fixed number of bits/bytes for digital representation, irrespective of the complexity of the chemical structure. The molecular descriptor may, for example, be an n-dimensional vector, where n is an integer, where n may assume, for example, the values of 128, 256, 512, 1024 or other values. Details of the molecular descriptor and of the generation thereof can be found further down in the description.

In some embodiments, in a first step, spectroscopic data of a chemical compound are received. In some embodiments, in a further step, the spectroscopic data are inputted into the first artificial neural network.

In some embodiments, such an artificial neural network comprises at least three layers of processing elements: a first layer with input neurons (nodes), an N-th layer with at least one output neuron (nodes) and N-2 inner layers, where N is a natural number and greater than 2.

In some embodiments, the input neurons of the first artificial neural network serve to receive the spectroscopic data.

In some embodiments, the output neurons of the first artificial neural network serve to output the molecular descriptor.

In some embodiments, the processing elements of the layers between the input neurons and the output neurons are connected to one another in a predetermined pattern with predetermined connection weights.

In some embodiments, the artificial neural network is configured to use spectroscopic data of a chemical compound to generate a molecular descriptor as output. The neural network is typically configured by means of a supervised learning method.

In some embodiments, the learning/training is effected with the aid of training data of a multitude of chemical reference compounds. The training data may include, for each chemical reference compound of the multitude of chemical reference compounds, spectroscopic data (input vector) and a molecular descriptor (output vector).

In some embodiments, the training of the neural network can, for example, be carried out by means of a backpropagation method. The aim here in respect of the network is maximum reliability of mapping of given input vectors onto given output vectors. The mapping quality is described by an error function. The goal is to minimize the error function. In the case of the backpropagation method, an artificial neural network is taught by the alteration of the connection weights.

In the trained state, the connection weights between the processing elements contain information relating to the relationship between spectroscopic data of a chemical compound and the molecular descriptor of the chemical compound. This information can be used to predict molecular descriptors for new chemical compounds on the basis of the spectroscopic data thereof. The term “new” here is understood to mean the spectroscopic data of a chemical compound have not already been used in the training of the neural network. The neural network is thus capable of applying the learned “knowledge” to other chemical compounds.

In some embodiments, a cross-validation method can be used in order to divide the training data into training and validation data sets. The training data set may be used in the backpropagation training of network weights. The validation data set may be used in order to check the accuracy of prediction with which the trained network can be applied to unknown (new) chemical compounds (or the spectroscopic data thereof).

Details of the construction and of the generation of artificial neural networks can be found in the prior art (see, for example: R. Rojas: Neural Networks: A Systematic Introduction, Springer-Verlag 2013, ISBN: 9783642610684; D. Graupe: Principles Of Artificial Neural Networks: Basic Designs To Deep Learning, 4th edition, World Scientific Publishing, 2019, ISBN: 9789811201240).

The spectroscopic data used to train the first artificial neural network may be measured and/or calculated spectroscopic data. Spectroscopic data for a multitude of chemical compounds (reference compounds) can be found, for example, in publicly accessible databases (see, for example: https://pubchem.ncbi.nlm.nih.gov). Software tools for calculation of NMR spectra are commercially and freely available (see, for example: https://www.nmrdb.org; http://www.cheminfo.org/; http://www.colby.edu/chemistry/NMR/scripts/C13chemshift.html).

In some embodiments, in a further step, the molecular descriptor generated by the first artificial neural network is inputted into the second neural network. It is also possible here for the first neural network and the second neural network to be directly linked to one another; the output neurons from the first network may be directly associated with or even identical to the input neurons for the second neural network. The reason why this description nevertheless refers to two neural networks, the first neural network and the second neural network, is that the first neural network and the second neural network are typically configured (trained) independently of one another. As soon as they are configured (trained), they may be combined to form one network. The present invention is thus not to be understood in such a way that the first and second neural networks must necessarily be separated from one another in the prediction of structure data based on spectroscopic data; instead, the invention is to be understood in such a way that there may also be a single network for prediction that is composed of two independently configured (trained) sub-networks.

In some embodiments, the second artificial neural network is a decoder of an autoencoder. An autoencoder consists of two artificial neural networks (sub-networks): an encoder and a decoder. The aim of an autoencoder is typically to learn a compressed representation (encoding) for a set of data and hence to extract essential features. In this way, it can be used to reduce the dimensions.

In some embodiments, the encoder converts structure data of variable length as input sequence to a molecular descriptor of fixed size. The decoder converts the molecular descriptor (back) into the sequence of structure data.

In some embodiments, the autoencoder is typically trained in an unsupervised learning method to minimize the reconstruction error at the individual character level for each input sequence. The training can be automated, i.e. the autoencoder can learn in an automated manner using structure data of a multitude of chemical reference compounds, transform these structure data of variable length into codes of fixed size (molecular descriptors), and reconstruct the structure data from the molecular descriptors.

Structure data of a multitude of chemical reference compounds can be taken from databases, for example PubChem (see, for example: https://pubchem.ncbi.nlm.nih.gov/; K. Sunghwan et al.: PubChem 2019 update: improved access to chemical data, Nucleic Acids Research, Volume 47, Issue D1, 2019, D1102-D1109, https://doi.org/10.1093/nar/gky1033) and/or Zinc (see, for example: http://zinc15.docking.orgi; J. J. Irwin et al.: ZINC—a free database of commercially available compounds for virtual screening, J Chem Inf Model, 2005;45(1):177-182, doi:10.1021/ci049714).

Further details of autoencoders and the generation thereof can be found, for example, in Q. V. Le: A Tutorial on Deep Learning Part 2: Autoencoders, Convolutional Neural Networks and Recurrent Neural Networks, 2015, https://cs.stanford.edu/˜quocle/tutorial2.pdf; W. Meng: Relational Autoencoder for Feature Extraction, arXiv:1802.03145v1; WO2018046412A1).

The autoencoder used is preferably based on the architecture published by Winter et al. (see R. Winter et al.: Chem. Sci., 2019, 10, 1692-1701; Chem. Sci., 2020, 11, 10378-10389).

In some embodiments, the molecular descriptors are continuous and data-driven molecular descriptors (cddd), as described, for example, in the following publications: T. Le, R. Winter, F, Noé, D.-A. Clevert: Neuraldecipher—reverse-engineering extended-connectivity fingerprints (ECFPs) to their molecular structures, Chem. Sci., 2020, 11, 10378; R. Winter, F. Montanan, F. Noé, D.-A. Clevert: Learning continuous and data-driven molecular descriptors by translating equivalent chemical representations, Chem. Sci., 2019, 10, 1692).

In some embodiments, in a further step, structure data of the chemical compound are received as output from the second artificial neural network. The structure data and/or information derived therefrom may, in a further step, be displayed on a display screen, printed out and/or stored in a data storage medium.

If the structure data are, for example, a structure code for a chemical compound, it is possible (as well as or instead of the structure data) to display an image representation of the chemical structure of the chemical compound on a display screen, to print it out and/or to store it in a data storage medium. Such an image representation of the chemical structure may, for example, be an electronic formula, valence bond formula, perspective bond formula and/or skeletal formula (for representation of chemical structures see, for example, J. K. Felixberger: Chemie für Einsteiger [Chemistry for Beginners], Springer-Verlag 2017, ISBN: 978-3-662-52824-0, chapter 5.5 and S. Feil et al.: Faszinierende Chemie [Fascinating Chemistry], Springer-Verlag 2017, ISBN: 978-3-662-49919-1, pages 32-33 and E. Benfenati et al.: Characterization of chemical structures, Chapter 3 of Quantitative Structure—Activity Relationships (QSAR) for Pesticide Regulatory Purposes, Elsevier-Verlag 2007, ISBN: 978-0-444-52710-3, pages 83 to 109).

In some embodiments, spectroscopic data are calculated (for example a ¹³C NMR spectrum and/or a H NMR spectrum) for the chemical compound, the structure data of which have been predicted in accordance with the invention from spectroscopic data, and these spectroscopic data are compared to those spectroscopic data that have been used for the prediction. Thus, the result of the prediction can be verified and assessed directly by comparing the two spectroscopic data sets to one another. In the case of a correct prediction, the two sets of spectroscopic data correspond. In the case of an incorrect prediction, it is possible by identifying the deviations in the stereoscopic data sets to ascertain the structural features with which the first artificial neural network and/or the second artificial neural network are still having problems. The deviations can be used to find and/or select and/or generate chemical compounds for further (supplementary) training. Automated reinforced learning is also conceivable.

The present invention can be performed wholly or partly with a computer system. A “computer system” is a system for electronic data processing that processes data by means of programmable computation rules. Such a system usually comprises a control and computation unit, often also referred to as “computer”, said unit comprising a processor for carrying out logical operations and a memory for loading a computer program, and also a peripheral.

In computer technology, “peripherals” refers to all devices that are connected to the computer and are used for control of the computer and/or as input and output devices. Examples thereof are monitor (screen), printer, scanner, mouse, keyboard, joystick, drives, camera, microphone, speakers, etc. Internal ports and expansion cards are also regarded as peripherals in computer technology.

Modern computer systems are frequently divided into desktop PCs, portable PCs, laptops, notebooks, netbooks and tablet PCs, and what are called handhelds (e.g. smartphones); all these systems can be utilized for execution of the invention.

Inputs into the computer system (e.g. for control by a user) are achieved via input means such as, for example, a keyboard, a mouse, a microphone, a touch-sensitive display and/or the like. Outputs are achieved via one or more output units, which may be especially a monitor (screen), a printer and/or a data storage medium.

The invention is elucidated in detail hereinafter with reference to drawings, without any intention to restrict the invention to the features or combinations of features shown in the figures.

### BRIEF DESCRIPTION OF THE DRAWINGS

FIG. 1 shows a computer system, according to some embodiments of the present disclosure.

FIG. 2 shows an example of an image representation of the chemical structure of a chemical compound.

FIG. 3 shows a ¹³C NMR spectrum of the chemical compound shown in FIG. 2.

FIG. 4 shows a method for generating codes for chemical structures, according to some embodiments.

FIG. 5 shows the mode of function of the first artificial neural network, according to some embodiments.

FIG. 6 shows the mode of function of an autoencoder, according to some embodiments.

FIG. 7 shows the interplay of the first and second artificial neural networks in the generation of structure data, according to some embodiments.

### DETAILED DESCRIPTION OF EXEMPLARY EMBODIMENTS

FIG. 1 shows, in schematic form a computer system according to some embodiments of the present disclosure. The computer system (10) comprises an input unit (11), a control and computation unit (12) and an output unit (13).

In some embodiments, the computer system (10) may be configured to receive spectroscopic data of chemical compounds, to use the spectroscopic data received to generate structure data for the chemical compounds, and to output and/or to store the structure data and/or information derived therefrom.

In some embodiments, the control and computation unit (12) may serve to control the input unit (11) and the output unit (13), to coordinate the flows of data and signals between the different units, to process spectroscopic data and further data, and to create structure data based on the spectroscopic data by means of a first and second artificial neural network. In some embodiments, the first and second artificial neural network may be loaded, for example, in a memory of the computer system that may be part of the control and computation unit (12).

In some embodiments, the input unit (11) may serve to receive spectroscopic data of chemical compounds. In some embodiments, the spectroscopic data may be provided/transmitted, for example, via a network (not shown in FIG. 1) by another computer system and/or read out from a database that may be part of the computer system according to the invention or may be connected thereto via a network.

In some embodiments, spectroscopic data can be transmitted via a network connection or a direct connection. Spectroscopic data can be transmitted via radio communication (WLAN, Bluetooth, mobile communications and/or the like) and/or via a cable. It is conceivable that multiple input units are present.

In some embodiments, the input unit (11) may transmit the spectroscopic data and any further data to the control and computation unit (12). In some embodiments, the control and computation unit (12) may be configured to generate structure data using the data received.

In some embodiments, the output unit (13) can display the structure data and/or information derived therefrom (for example on a monitor), output them (for example via a printer) or store them in a data storage medium.

It is conceivable that multiple output units are present. It is likewise possible for there to be multiple input units and/or control and/or computation units.

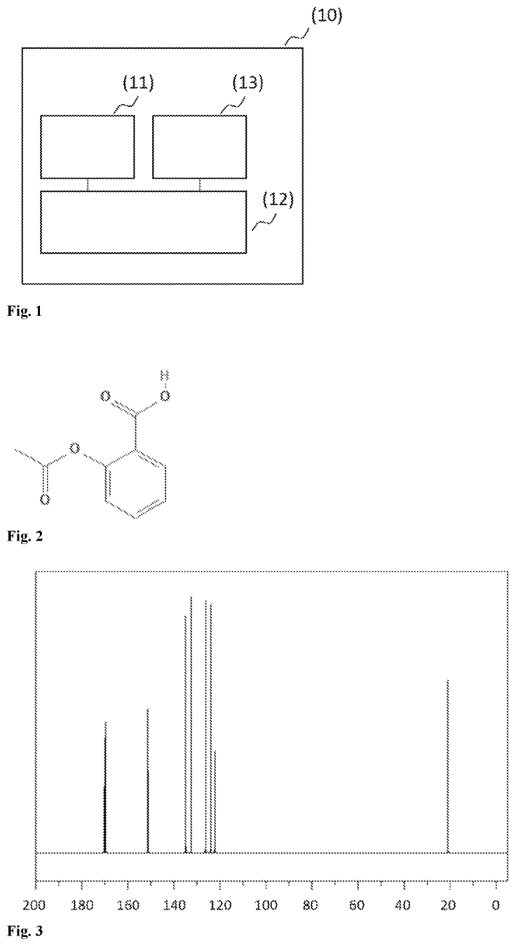

FIG. 2 shows an example of an image representation of the chemical structure of a chemical compound. The compound is acetylsalicylic acid; the IUPAC name of the compound is 2-acetoxybenzoic acid. The structure represented as an image is a skeletal formula: the carbon atoms and the hydrogen atoms bonded to carbon atoms are not shown explicitly; all that are shown explicitly are the oxygen atoms (O) and the hydrogen atom (H) bonded to an oxygen atom by their element symbols (O, H).

The SMILES code for 2-acetoxybenzoic acid is: CC(═O)OCl═CC═CC═ClC(═O)O.

The InChI code for 2-acetoxybenzoic acid is: 1S/C9H8O4/cl-6(10)13-8-5-3-2-4-7(8)9(11)12/h2-5H,1H3,(H,11,12).

The SMILES code and the InChI code are examples of chemical structure codes.

FIG. 3 shows, by way of example, a ¹³C NMR spectrum of the chemical compound shown in FIG. 2 (2-acetoxybenzoic acid). The spectrum was recorded in deuterochloroform as solvent (0.039 g of 2-acetoxybenzoic acid in 0.5 ml of CDCl₃) at 50.18 MHz. The chemical shift δ in ppm is given on the abscissa.

One example of a peak list for a calculated ¹³C NMR spectrum (in this case for 2-acetoxybenzoic acid) is:
δ (ppm)
20.77529
120.91842
122.26
124.01
131.33934
133.14333
151.28
166.94
169.22

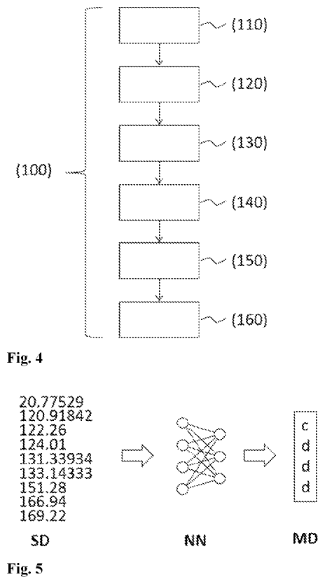

FIG. 4 shows a method according to some embodiments of the present disclosure in the form of a flow chart.

In some embodiments, the process (100) comprises the steps of:
(110) receiving spectroscopic data of a chemical compound,
(120) inputting the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
(130) receiving a molecular descriptor of the chemical compound from the first artificial neural network,
(140) inputting the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
(150) receiving structure data of the chemical compound from the second artificial neural network,
(160) outputting and/or storing the structure data and/or information derived from the structure data.

FIG. 5 shows the mode of function of the first artificial neural network, according to some embodiments. In some embodiments, the first neural network (NN) may be trained to use spectroscopic data (SD) as input to generate a molecular descriptor (MD). In some embodiments, it may use a peak list of a ¹³C NMR spectrum to generate a continuous and data-driven molecular descriptor (cddd).

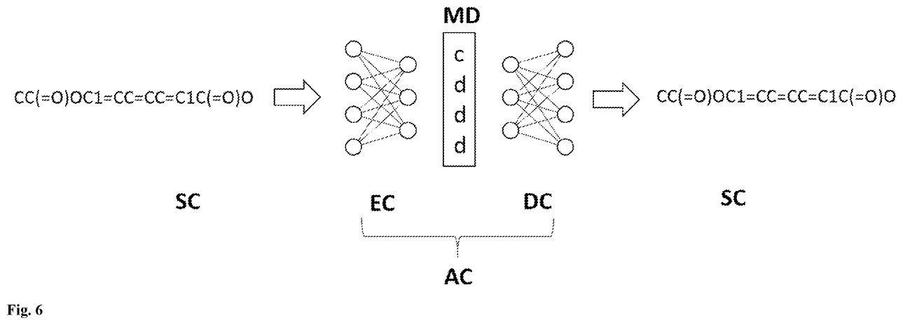

FIG. 6 shows the mode of function of an autoencoder, according to some embodiments. In some embodiments, the autoencoder (AC) may consist of an encoder (EC) and a decoder (DC). In some embodiments, the autoencoder (AC) may be trained to use structure data, in the present case from a chemical structure code of a chemical compound, to generate a molecular descriptor (MD) (encoding), and to use the molecular descriptor (MD) to reconstruct the chemical structure code (decoding). In the present case, the encoder (EC) of the autoencoder (AC) uses the SMILES code of 2-acetoxybenzoic acid (CC(═O)OCl═CC═CC═ClC(═O)O) to generate a continuous and data-driven molecular descriptor (cddd), and the decoder (DC) of the autoencoder (AC) uses the continuous and data-driven molecular descriptor (cddd) to generate the SMILES code of 2-acetoxybenzoic acid (CC (═O)OCl═CC═CC═ClC(═O)O).

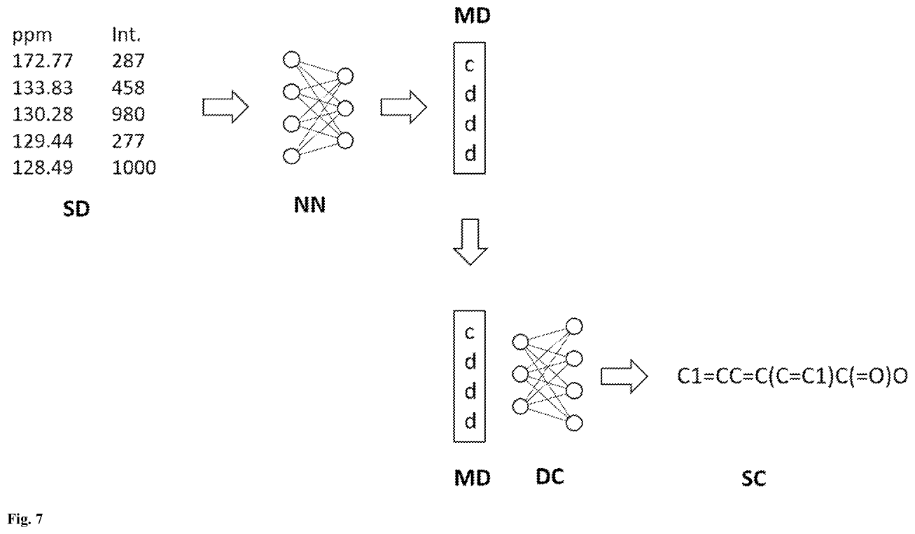

FIG. 7 shows the interplay of the first and second artificial neural networks in the generation of structure data of a chemical structure of a chemical compound from spectroscopic data of the chemical compound, according to some embodiments. The first artificial neural network (NN) shown in FIG. 7 is the network shown in FIG. 5. The second artificial neural network (DC) shown in FIG. 7 is the decoder of the autoencoder (AC) shown in FIG. 6. In some embodiments, the first artificial neural network (NN) may receive spectroscopic data of a chemical compound (in the present case of benzoic acid). In some embodiments, the first artificial neural network (NN) may use the spectroscopic data to generate a molecular descriptor (MD), in the present example a continuous and data-driven molecular descriptor (cddd). This continuous and data-driven molecular descriptor (cddd) is sent to the second artificial neural network (DC). In some embodiments, the second artificial neural network (DC) may use the molecular descriptor to generate the SMILES code of benzoic acid (Cl═CC═C(C═Cl)C(═O)O).

Table 1 below shows images of a series of chemical structures of chemical compounds in the form of their skeletal formulae (middle column). The left-hand column of Table 1 gives a peak list of a ¹³C NMR spectrum for each of the chemical compounds. The right-hand column of Table 1 lists the SMILES codes predicted in accordance with the invention for the chemical compounds on the basis of the peak lists. For the prediction of the SMILES codes, a multilayer feedforward neural network with fully connected layers was used as first neural network, and the decoder of an autoencoder based on the architecture described by Winter et al. as second neural network (Chem. Sci., 2019, 10, 1692-1701; Chem. Sci., 2020, 11, 10378-10389).

TABLE 1

| | | |
|---|---|---|
| 14.3; 0.0Q; 9\|14.3; 0.0Q; 12\|24.8; 0.0Q; 5\|24.8; 0.0Q; 6\|61.4; 0.0T; 8\|61.4; 0.0T; 11\|123.1; 0.0S; 1\|123.1; 0.0S; 4\|141.0; 0.0D; 0\|162.1; 0.0S; 2\|162.1; 0.0S; 3\|165.7; 0.0S; 7\|165.7; 0.0S; 10 | 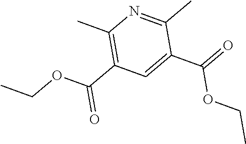 [SMILES: CCOC(=O)c1cc(C(=O)OCC)c(C)nc1C] | CCOC(═O)c1cc(C(═O)OCC)c(C)nc1C |
| 15.1; 0.0Q; 10\|15.1; 0.0Q; 12\|60.9; 0.0T; 9\|60.9; 0.0T; 11\|84.4; 0.0S; 6\|85.1; 0.0S; 7\|91.8; 0.0D; 8\|121.9; 0.0S; 3\|128.2; 0.0D; 1\|128.2; 0.0D; 5\|128.7; 0.0D; 0\|131.8; 0.0D; 2\|131.8; 0.0D;4 | 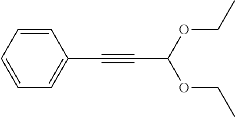 | CCOC(C#Cc1ccccc1)OCC |
| 14.6; 0.0Q; 7\|14.6; 0.0Q; 9\|62.2; 0.0T; 6\|62.2; 0.0T; 8\|101.9; 0.0S; 0\|102.3; 0.0D; 5\|109.8; 0.0D; 2\|113.3; 0.0S; 4\|121.2; 0.0D; 1\|123.3; 0.0D; 3 | 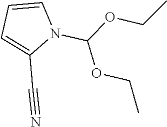 | CCOC(OCC)n1cccc1C#N |
| 52.32; 0.0Q; 9\|69.56; 0.0T; 6\|69.56; 0.0T; 7\|106.91; 0.0D; 3\|107.87; 0.0D; 1\|107.87; 0.0D; 5\|127.7; 0.0D; 11\|127.7; 0.0D; 15\|127.7; 0.0D; 17\|127.7; 0.0D; 21\|127.92; 0.0D; 13\|127.92; 0.0D; 19\|128.46; 0.0D; 12\|128.46; 0.0D; 14\|128.46; 0.0D; 18\|128.46; 0.0D; 20\|131.61; 0.0S; | 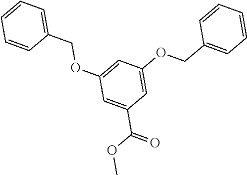 [SMILES: COC(=O)c1cc(OCc2ccccc2)cc(OCc2ccccc2)c1] | COC(═O)c1cc(OCc2ccccc2)cc(OCc2ccccc2)c1 |
| 0\|136.64; 0.0S; 10\|136.64; | | |
| 0.0S; 16\|159.51; 0.0S; | | |
| 2\|159.51; 0.0S; 4\|165.83; | | |
| 0.0S; 8 | | |
| 26.8; 0.0Q; 9\|124.4; 0.0D; 0\|124.4; 0.0D; 3\|131.0; 0.0S; 4\|131.0; 0.0S; 5\|135.5; 0.0D; 1\|135.5; 0.0D; 2\|165.3; 0.0S; 6\|165.3; 0.0S; 7\|168.8; 0.0S;8 | 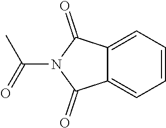 | CC(═O)N1C(═O)c2ccccc2C1═O |

1. A computer-implemented method, the method comprising:
receiving spectroscopic data of a chemical compound,
inputting the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
receiving a molecular descriptor of the chemical compound from the first artificial neural network,
inputting the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
receiving structure data of the chemical compound from the second artificial neural network, and
outputting and/or storing the structure data and/or information derived from the structure data.

2. The method of claim 1, wherein the spectroscopic data are data from a nuclear resonance spectrum or multiple nuclear resonance spectra of the chemical compound.

3. The method of claim 1, wherein the spectroscopic data are a peak list from a ¹³C NMR spectrum and/or a ¹H NMR spectrum.

4. The method of claim 1, wherein the molecular descriptor is an n-dimensional vector.

5. The method of claim 1, 4, wherein the molecular descriptor is a continuous and data-driven molecular descriptor.

6. The Method according to any of method of claim 1, wherein the structure data are a chemical structure code.

7. The method of claim 1, wherein the structure data are a SMILES, InChI, CML or WLN code.

8. The method of claim 1, to further comprising:
calculating spectroscopic data from the structure data received,
comparing the spectroscopic data calculated with the spectroscopic data received,
identifying the deviations between the spectroscopic data calculated and the spectroscopic data received, and
outputting and/or storing the deviations.

9. A system comprising a computer configured to
prompt the receipt of spectroscopic data of a chemical compound,
input the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
receive a molecular descriptor for the chemical compound from the first artificial neural network,
input the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
receive structure data of the chemical compound from the second artificial neural network,
prompt the output of and/or to store the structure data received and/or information derived from the structure data.

10. A non-transitory computer-readable storage medium comprising instructions that, when executed by a processor, cause the processor to:
receive spectroscopic data of a chemical compound,
input the spectroscopic data into a first artificial neural network, wherein the first artificial neural network has been trained, in a supervised learning method using spectroscopic data of a multitude of chemical reference compounds, to generate molecular descriptors of the chemical reference compounds on the basis of the spectroscopic data of the chemical reference compounds,
receive a molecular descriptor of the chemical compound from the first artificial neural network,
input the molecular descriptor received into a second artificial neural network, wherein the second artificial neural network is a decoder of an autoencoder, wherein the autoencoder has been trained, in an unsupervised learning method using a multitude of chemical reference compounds, to convert structure data of the chemical reference compounds to molecular descriptors and to convert the molecular descriptors back to the structure data,
receive structure data of the chemical compound from the second artificial neural network,
output and/or store the structure data and/or information derived from the structure data.

11. The non-transitory computer readable storage medium of claim 10, wherein the instructions prompt the processor to execute one or more of:
calculating spectroscopic data from the structure data received;
comparing the spectroscopic data calculated with the spectroscopic data received;
identifying the deviations between the spectroscopic data calculated and the spectroscopic data received; and
outputting and/or storing the deviations.
